(19) United States
(12) Patent Application Publication
(10) Pub. No.: US 2026/0355188 A1
(43) Pub. Date: Nov. 12, 2026

(54) COMBINATION THERAPIES USING PRMT5 INHIBITORS AND BCL-2 FAMILY INHIBITORS FOR THE TREATMENT OF CANCER

(71) Applicant: MIRATI THERAPEUTICS, INC., PRINCETON, NJ (US)

(72) Inventors: LARS DANIEL ENGSTROM, ESCONDIDO, CA (US); PETER OLSON, SAN DIEGO, CA (US); LAURA WATERS, SAN DIEGO, CA (US); VICKIE BOWCUT, SAN DIEGO, CA (US)

(73) Assignee: MIRATI THERAPEUTICS, INC., PRINCETON, NJ (US)

(21) Appl. No.: 19/107,765

(22) PCT Filed: Aug. 31, 2023
(86) PCT No.: PCT/US2023/031608

§ 371 Date: Feb. 28, 2025

Related U.S. Application Data
(60) Provisional application No. 63/403,201, filed on Sep. 1, 2022.
(60) Provisional application No. 63/497,022, filed on Apr. 19, 2023.

(51) Int. Cl. A61K 31/502; A61K 31/635; A61P 35/00

(52) U.S. Cl. CPC A61K 31/502; A61K 31/635; A61P 35/00

(57) ABSTRACT

Disclosed herein are methods of treating cancer. More specifically, this disclosure provides methods for treating cancer in a subject using compounds that are inhibitors of PRMT5, particularly in combination with a BCL-2 family inhibitor.

### BACKGROUND OF THE DISCLOSURE

### Cross-Reference to Related Applications

This application claims priority from U.S. Provisional Application No. 63/403,201, filed Sep. 1, 2022, and U.S. Provisional Application No. 63/497,022, filed Apr. 19, 2023, the disclosure of each of which is hereby incorporated by reference in its entirety.

### FIELD OF THE DISCLOSURE

This disclosure relates to methods of treating cancer. This disclosure further relates to methods of treating cancer in a subject with compounds that are inhibitors of protein arginine N-methyl transferase 5 (PRMT5) in combination with a BCL-2 family inhibitor, particularly in combination with a BCL-2 family inhibitor having activity against BCL-xL.

### DESCRIPTION OF RELATED ART

PRMT5 is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with methylosome protein 50 (MEP50), which is required for substrate recognition and orientation and is also required for PRMT5-dependent SDMA modification of histone 2A and histone 4 (e.g., see Ho et al. (2013) PLoS ONE 8 (2): e57008).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139 (39): 13754-13760).

Cells lacking MTAP activity have elevated levels of the MTAP substrate, methylthioadenosine (MTA), which is a potent inhibitor of PRMT5. Inhibition of PRMT5 activity from elevated MTA results in reduced methylation activity and increased sensitivity of cellular proliferation to further PRMT5 depletion or inhibition of activity. Hence, the loss of MTAP activity reduces the methylation activity of PRMT5 making the cells selectively dependent on PRMT5 activity.

Despite importance of PRMT5 on cell viability and its prevalence in cancers, effective therapies that inhibit PRMT5 have been elusive. Thus, there remains a need to develop new PRMT5 inhibitor therapies to treat wide range of cancers.

BCL-2 family inhibitors are being investigated for the treatment of cancers. BCL-2 family inhibitors are hypothesized to work by inhibiting pro-survival signaling mediated through select BCL-2 family members. Inhibition of BCL-2 family members are anticipated to block the survival of cancer cells and provide clinical benefit to cancer patients. The Bcl-2 inhibitor venetoclax is approved for the treatment of chronic lymphocytic leukemia. The Bcl-2, Bcl-xL and Bcl-w inhibitor navitoclax is currently in clinical trials for a number of liquid and solid cancers. Navitoclax is being investigated as a single agent and in combination with therapies including chemotherapy. The Bcl-xL inhibitor DT2216 is being investigated for the treatment of various solid and liquid cancers. BCL-2 family inhibitors are anticipated to have differential utility for the treatment of various cancers depending on the various cancer types and the sensitivity of those cancers to inhibition of one or more BCL-2 family member.

Despite the importance of BCL-2 family members for cancer cell survival and its prevalence in resistance to cancer treatments, effective therapies that inhibit BCL-2 family members have been elusive. Thus, there remains a need to improve the efficacy of cancer therapies involving administration of BCL-2 family inhibitors.

### SUMMARY OF THE DISCLOSURE

The disclosure provides methods for treating cancer in a subject. Such methods include administering to the subject a therapeutically effective amount of a PRMT5 inhibitor in combination with a therapeutically effect amount of a BCL-2 family inhibitor. The BCL-2 family inhibitor in such combination and/or combination therapy may be selected from one or more of: ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax, DT2216, AMG176, PRT1419, AZD5991, S64315/MIK665, or combinations of these. Additional BCL-2 family inhibitors are currently being developed and can be used in embodiments of the invention herein described. The PRMT5 inhibitor may be, for instance, any of the PRMT5 inhibitors disclosed in WO 2021/050915 A1, including MRTX-1719.

Also provided herein is a method for treating cancer in a subject identified as being in need thereof. Such methods include a step of determining whether/that the subject's cancer is associated with MTAP homozygous deletion (e.g., an MTAP-associated cancer).

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods of the disclosure and are incorporated in and constitute a part of this specification. The drawings illustrate one or more embodiment(s) of the disclosure and, together with the description, serve to explain the principles and operation of the disclosure.

FIG. 1 illustrates the results of Example 1, wherein MRTX1719 (100 mg/kg PO, QD), the BCL-2 selective inhibitor venetoclax (ABT-199) (100 mg/kg PO, QD) or the combination; or wherein MRTX1719 (100 mg/kg PO, QD), the BCL-2/BCL-xL inhibitor navitoclax (ABT-263) (100 mg/kg PO, QD) or the combination; were dosed to mice bearing LU99 xenograft tumors (n=5/cohort). Data shown as mean tumor volume+/−SEM.

FIG. 2 illustrates the results of Example 2, wherein MRTX1719 (100 mg/kg PO, QD), the BCL-2/Bcl-w/BCL-xL inhibitor navitoclax (ABT-263) (100 mg/kg PO, QD) or the combination; or wherein MRTX1719 (100 mg/kg PO, QD), the BCL-xL selective inhibitor A-1331852 (25 mg/kg PO, QD) or the combination; were dosed to mice bearing NCI-H1437 xenograft tumors (n=4/cohort). Data shown as mean tumor volume+/−SEM.

FIG. 3 illustrates the results of Example 3, wherein MRTX1719 (100 mg/kg PO, QD), the BCL-2/Bcl-w/BCL-xL inhibitor navitoclax (ABT-263) (100 mg/kg PO, QD) or the combination; or wherein MRTX1719 (100 mg/kg PO, QD), the BCL-xL selective inhibitor A-1331852 (25 mg/kg PO, QD) or the combination; were dosed to mice bearing SW1573 xenograft tumors (n=4/cohort). Data shown as mean tumor volume+/−SEM.

FIG. 4 illustrates the results of Example 4, wherein MRTX1719 (100 mg/kg PO, QD), the BCL-xL selective inhibitor A-1331852 (25 mg/kg PO, BID) or the combination; were dosed to mice bearing LU99 xenograft tumors (n=5/cohort). Data shown as mean tumor volume+/−SEM.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. The present disclosure provides improvements in treating cancer in a subject. As used herein, the terms “subject” or “patient” are used interchangeably, refers to any animal, including mammals, and most preferably humans.

The PRMT5 inhibitors of the disclosure demonstrate selective activity in MTAP-deleted cancers by binding to and further inhibiting PRMT5 when bound to the intracellular metabolite MTA. As noted above, MTAP is an enzyme in the methionine salvage pathway and its deletion in cancer cells leads to the accumulation of MTA in these cells. PRMT5 is an essential enzyme required for cell viability and, as such, the PRMT5 inhibitors of the disclosure represent a novel approach to selectively treat MTAP-deleted cancers.

A single mutation will likely not cause cancer-most often, it is multiple mutations that are responsible for developing cancer. The inventors found the treatment of certain cancers with PRMT5 inhibitors improved with the use of combination therapies. Particularly, the inventors surprisingly found that a combination therapy of an MTA-cooperative PRMT5 inhibitor and a BCL-2 family inhibitor (e.g., ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax, and/or DT2216) inhibits in vitro tumor cell viability in a synergistic fashion and provides greater in vivo tumor growth inhibition compared to either inhibitor alone.

Without wishing to be bound by theory, the present inventors have observed that PRMT5 inhibition, such as by PRMT5 inhibitors as otherwise described herein, likely induce cell death in cancerous tissues through DNA damage. Accordingly, it was hypothesized that the provision of an additional therapeutic agent that enhances apoptosis, or programed cell death, may serve to enhance the therapeutic effect. In certain embodiments, for example, a BCL-2 family inhibitor such as ABT-199 (venetoclax), ABT-263 (navitoclax) or A-1331852 was administered in combination with a PRMT5 inhibitor. As disclosed herein, the in vitro combination was surprisingly found to effectively inhibit tumor cell viability in a synergistic fashion. As disclosed herein, the in vivo combination was surprisingly found to effectively inhibit tumor volume to a greater degree than either single agent alone.

The methods provided herein may be used for the treatment of a wide variety of cancer including tumors such as lung, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments of the methods of the disclosure, the cancer is a MTAP-associated cancer. For example, in certain embodiments, the cancer comprises MTAP gene homozygous deletion (MTAP^{DEL}). The subject may be identified or diagnosed as having MTAP-associated cancer where, for example, MTAP^{DEL} is determined using a suitable assay or a kit. Alternatively, the subject is suspected of having MTAP-associated cancer or the subject has a clinical record indicating that the subject has MTAP-associated cancer.

In some embodiments of any of the methods or uses described herein, an assay is used to determine subject treatment eligibility using a sample (e.g., a biological sample or a biopsy sample such as a paraffin-embedded biopsy sample) from a subject. Such assay includes, but is not limited to, next generation sequencing, next generation sequencing of circulating tumor DNA (ctDNA) in plasma, immunohistochemistry, fluorescence microscopy, break apart FISH analysis, Southern blotting, western blotting, FACS analysis, and PCR-based amplification (e.g., RT-PCR and quantitative real-time RT-PCR). As is well known in the art, the assays are typically performed, e.g., with at least one labelled nucleic acid probe or at least one labelled antibody or antigen-binding fragment thereof.

In certain embodiments, the cancer in the methods of the disclosure is selected from non-small cell lung cancer, pancreatic cancer, head and neck cancer, bladder cancer, esophageal cancer, diffuse large B cell lymphoma, stomach cancer, melanoma, breast cancer, cholangiocarcinoma, mesothelioma, and malignant peripheral nerve sheath tumors.

In certain embodiments, the cancer in the methods of the disclosure is selected from lung cancer (e.g., mesothelioma or non-small cell lung cancer (NSCLC) including adenocarcinoma and squamous cell), pancreatic cancer, colon cancer, head and neck cancer (such as squamous cell carcinoma (HNSCC)), bladder cancer, esophageal cancer, lymphoma (e.g., diffuse large B-cell lymphoma), stomach cancer, melanoma, breast cancer, and brain cancer (e.g., glioblastoma multiforme and glioma).

In certain embodiments, the cancer in the methods of the disclosure is selected from lung cancer (e.g., mesothelioma or NSCLC, including adenocarcinoma and squamous cell), pancreatic cancer, colon cancer, head and neck cancer (e.g. squamous cell carcinoma (HNSCC)), esophageal cancer, and melanoma.

In certain embodiments, the cancer in the methods of the disclosure is selected from mesothelioma, NSCLC (e.g., adenocarcinoma and squamous cell), pancreatic cancer, HNSCC, and colon cancer.

In one embodiment of the methods of the disclosure, the cancer is lung cancer. For example, the lung cancer may be NSCLC (e.g., adenocarcinoma and squamous cell) or mesothelioma. In certain embodiment, the cancer is NSCLC.

In one embodiment of the methods of the disclosure, the cancer is pancreatic cancer.

In one embodiment of the methods of the disclosure, the cancer is colon cancer.

In certain embodiments as otherwise described herein, the BCL-2 family inhibitor comprises at least one of: the BCL-2 selective inhibitor venetoclax (ABT-199), the BCL-2/Bcl-w/BCL-xL inhibitor navitoclax (ABT-263), A-1155463, A-1331852, obatoclax (GX15-070), ABT-737 TW-37 gossypol and (R)-(−)-gossypol, HA14-1, sabutoclax, DT2216, or combinations of these. For example, in particular embodiments, the BCL-2 family inhibitor is navitoclax. In other embodiments the BCL-2 family inhibitor is venetoclax. In further embodiments, the BCL-2 family inhibitor is A-1155463. In still further embodiments the BCL-2 family inhibitor is A-1331852. In an additional embodiment the BCL-2 family inhibitor is obatoclax. In an additional embodiment the BCL-2 family inhibitor is ABT-737. In an additional embodiment the BCL-2 family inhibitor is gossypol or (R)-(−)-gossypol. In an additional embodiment the BCL-2 family inhibitor is HA14-1. In another embodiment the BCL-2 family inhibitor is sabutoclax. In another embodiment the BCL-2 family inhibitor is DT2216.

As provided above, venetoclax (ABT-199) (CAS Registry Number: 1257044-40-8), navitoclax (ABT-263) (CAS Registry Number: 923564-51-6), A-1155463 (CAS Registry Number: 1235034-55-5), A-1331852 (CAS Registry Number: 1430844-80-6), obatoclax (GX15-070) (CAS Registry Number: 803712-67-6), ABT-737 (CAS Registry Number: 852808-04-9), TW-27 (CAS Registry Number: 877877-35-5), gossypol (CAS Registry Number: 303-45-7), ((R)-(−)-gossypol acetic acid (CAS Registry Number: 866541-93-7), HA 14-1 (CAS Registry Number: 65673-63-4), DT2216 (CAS Registry Number: 2365172-42-3) and/or sabutoclax (CAS Registry Number: 1228108-65-3) are administered in the methods of the disclosure. For example, venetoclax is an approved drug which is administered orally. Navitoclax is an unapproved drug which is administered orally. A-1155463 has been administered via intraperitoneal injection (IP) in in vivo studies. A-1331852 is known to be bioavailable when administered orally. Obatoclax is administered orally, typically in its mesylate form (CAS Registry Number: 803712-79-0).

As provided above, the PRMT5 inhibitor is also administered in the methods of the disclosure. A “PRMT5 inhibitor” as used herein refers to compounds of the disclosure as described herein. These compounds are capable of negatively modulating or inhibiting all or a portion of the enzymatic activity of the PRMT5, particularly, in the presence of bound MTA in vitro or in vivo or in cells containing elevated levels of MTA. In certain embodiments, the PRMT5 inhibitor is an MTA-cooperative PRMT5 inhibitor.

In certain embodiments, the PRMT5 inhibitor of the disclosure is any one of the PRMT5 inhibitors disclosed in international patent application no. PCT/US20/50457 filed 11 Sep. 2020 (published as WO 2021/050915 A1 on 18 Mar. 2021), which application and publication are herein incorporated by reference in their entireties. In certain other embodiments, the PRMT5 inhibitor of the disclosure is any one of the PRMT5 inhibitors disclosed in international patent application no. PCT/US22/020056 filed 11 Mar. 2022 (published as WO2022192745A1 on Sep. 15, 2022), which application and publication are herein incorporated by reference in their entireties.

In certain other embodiments, the PRMT5 inhibitor of the disclosure is any one of the PRMT5 inhibitors disclosed in international patent application no. PCT/US22/035508 filed 29 Jun. 2022 (published as WO2023278564 on Jan. 5, 2023), which application and publication are herein incorporated by reference in their entireties.

In certain other embodiments, the PRMT5 inhibitor of the disclosure is any one of the PRMT5 inhibitors disclosed U.S. provisional application Nos. 63/276,479 filed 5 Nov. 2021 and 63/356,861, filed 29 Jun. 2022, which is incorporated herein by reference in its entirety.

For example, the PRMT5 inhibitor in the methods of the disclosure as described herein is a compound of Formula IIA, IIB or IIC (Embodiment 1):

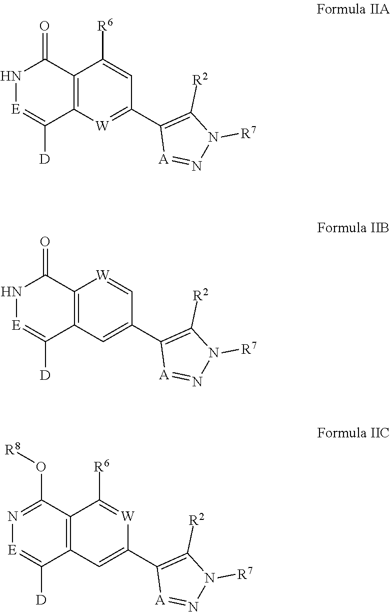

or a pharmaceutically acceptable salt thereof, wherein:
A is CR⁹ or N;
D is (C(R⁹)₂)₁₋₂—NH₂,

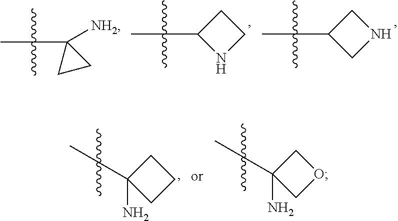

or D is

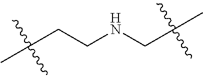 [SMILES: *CCNC*]

where the methylene is bonded to E where E is C;
E is C, CR⁹ or N;
each L is independently a bond or C₁-C₃ alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C₁-C₃alkyl, X—(C₁-C₃ alkyl)-aryl, heteroalkyl, C₂-C₄ alkynyl, —X-haloalkyl, —X—C₁-C₅ alkyl, -Z-C₁-C₅ alkyl, heterocyclyl, —X-L-cycloalkyl, -Z-cycloalkyl, —X-aryl, -Z-aryl, or —X-heteroaryl, wherein the heterocyclyl, the cycloalkyl, the aryl and the heteroaryl are optionally substituted with one or more R⁵;
each R⁴ is independently hydrogen or C₁-C₃ alkyl;
each R⁵ is independently cyano, oxo, halogen, C₁-C₃ alkyl, hydroxyalkyl, hydroxy, alkoxy, alkoxy-C₁-C₃ alkyl, —X-haloalkyl, -Z-cycloalkyl, X—(C₁-C₃ alkyl)-aryl, X—(C₁-C₃ alkyl)-aryl substituted with cyano, —X-L-cycloalkyl optionally substituted with C₁-C₃ alkyl or oxo, —X-L-heteroaryl optionally substituted with one or more C₁-C₃ alkyl or oxo, —X-L-heterocyclyl optionally substituted with one or more C₁-C₃ alkyl or oxo, or —X-aryl;
R⁶ is hydrogen, halogen, C₁-C₃ alkyl, haloalkyl, hydroxy, alkoxy, C₁-C₃ alkyl-alkoxy, N(R⁹)₂, NR⁹C(O)R⁹, C(O)R⁹, oxetane and THF;
R⁷ is H or C₁-C₃ alkyl optionally substituted with one or more halogen;
R⁸ is H or C₁-C₃ alkyl; and
each R⁹ is independently H or C₁-C₃ alkyl, halogen or haloalkyl.

Embodiment 2 provides the PRMT5 inhibitor in the methods of the disclosure as a compound of Formula IIA:

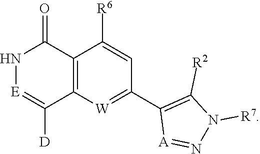

Embodiment 3 provides the PRMT5 inhibitor in the methods of the disclosure as a compound of Formula IIB:

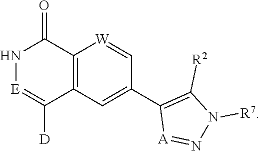

Embodiment 4 provides the PRMT5 inhibitor in the methods of the disclosure as a compound of Formula IIC:

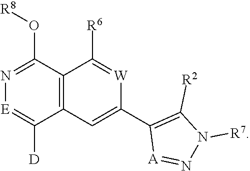

Embodiment 5 provides the method of any of embodiments 1-4, wherein W is CR⁹.

Embodiment 6 provides the method of any of embodiments 1-4, wherein A is CR⁹.

Embodiment 7 provides the method of any of embodiments 1-4, wherein E is N.

Embodiment 8 provides the method of any of embodiments 1-7, wherein W is CR⁹, A is CR⁹ and E is N.

Embodiment 9 provides the method of any of embodiments 1-8, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

Embodiment 10 provides the method of any of embodiments 1-8, wherein each R⁵ is independently cyano, oxo, halogen, C1-C3 alkyl, hydroxy, hydroxyalkyl, alkoxy-C1-C3alkyl, —X-L-heterocyclyl optionally substituted with one or more C1-C3alkyl or oxo, —X-L-cycloalkyl optionally substituted with C1-C3 alkyl or oxo.

Embodiment 11 provides the method of any of embodiments 1-8, wherein R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

Embodiment 12 provides the method of any of embodiments 1-11, where one of L, X and Z is a bond.

Embodiment 13 provides the method of embodiment 12, wherein all of L, X and Z are bonds.

One aspect of the disclosure provides the method wherein the PRMT5 inhibitor is a compound of the formula (IIIC) (Embodiment 14):

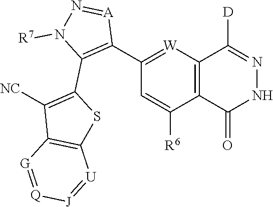

or a pharmaceutically acceptable salt thereof, wherein
A is CR⁹ or N;
   D is —CH₂—NH₂,

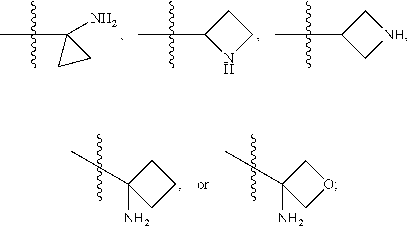 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, -N(R⁹)₂,
or —NR¹⁵(CO)R¹⁶,
   where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl.

Embodiment 15 provides the method according to embodiment 14, wherein A is CH.

Embodiment 16 provides the method according to embodiment 14 or 15, wherein W is N.

Embodiment 17 provides the method according to embodiment 14 or 15, wherein W is CH.

Embodiment 18 provides the method according to any of embodiments 14-17, wherein D is —CH₂—NH₂.

Embodiment 19 provides the method of the disclosure wherein the PRMT5 inhibitor is a compound according to embodiment 14 of the formula:

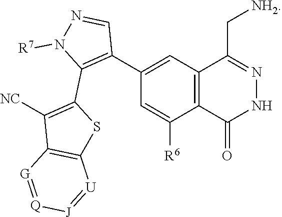

Embodiment 20 provides the method according to any of embodiments 14-19, wherein R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 21 provides the method according to any of embodiments 14-19, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 22 provides the method according to any of embodiments 14-19, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 23 provides the method according to any of embodiments 14-19, wherein R⁶ is halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 24 provides the method according to any of embodiments 14-19, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 25 provides the method according to any of embodiments 14-19, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 26 provides the method according to any of embodiments 23-25, wherein each G, Q, J and U is independently C(H).

Embodiment 27 provides the method according to any of embodiments 23-25, wherein G, Q, J and U are independently selected from C(H) and C(R⁵).

Embodiment 28 provides the method according to any of embodiments 23-25, wherein G, Q, J and U are independently selected from C(H) and N.

Embodiment 29 provides the method according to any of embodiments 14-19, wherein
R⁶ is hydrogen;
at least one of G, Q, J, and U is C(R⁵), and the remaining G, Q, J, and U are independently selected from C(H), C(R⁵) and N, wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 30 provides the method according to embodiment 29, wherein one or two of G, Q, J and U is N.

Embodiment 31 provides the method according to any of embodiments 14-19, wherein
R⁶ is hydrogen;
at least one of G, Q, J, and U is C(R⁵), and the remaining G, Q, J, and U are independently selected from C(H) and C(R⁵), wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 32 provides the method according to embodiment 31, wherein at least one of G, Q, J, and U is C(R⁵), and the remaining G, Q, J, and U are independently C(H); for example only one of G, Q, J, and U is C(R⁵).

Embodiment 33 provides the method according to embodiment 31, wherein two of G, Q, J, and U is C(R⁵), and the remaining G, Q, J, and U are independently C(H).

Embodiment 34 provides the method according to embodiment 31, wherein three of G, Q, J, and U is C(R⁵), and the remaining G, Q, J, and U is C(H).

Embodiment 35 provides the method according to any of embodiments 14-19, wherein G, Q, J, and U together with the thiophene to which they are attached form:

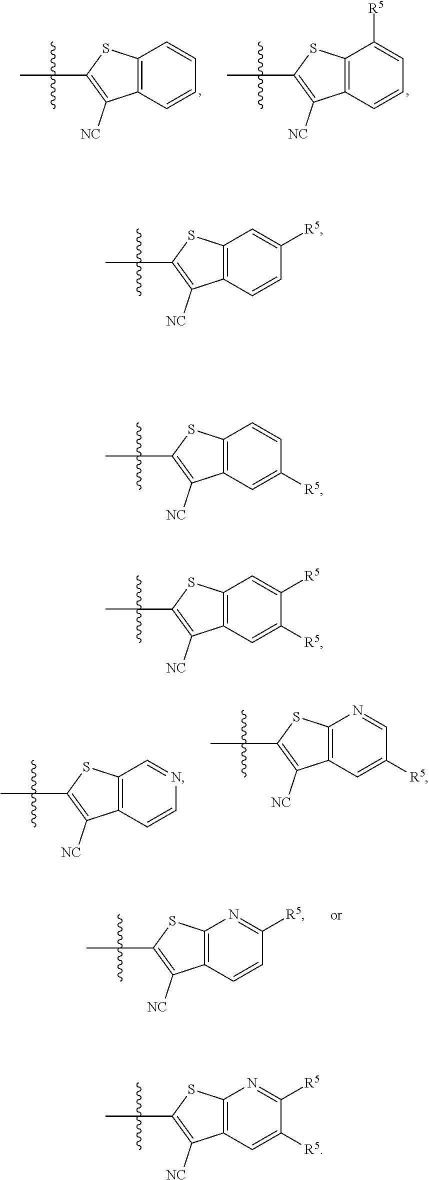

Embodiment 36 provides the method according to embodiment 35, wherein G, Q, J, and U together with the thiophene ring to which they are attached form a benzo[b]thiophene.

Embodiment 37 provides the method according to any one of embodiments 14-36, wherein R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 38 provides the method according to any one of embodiments 14-36, wherein R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 39 provides the method according to any one of embodiments 14-36, wherein R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

Embodiment 40 provides the method according to any one of embodiments 14-39, wherein R⁷ is methyl.

Embodiment 41 provides the method according to any one of embodiments 14-39, wherein R⁷ is ethyl.

Embodiment 42 provides the method according to any one of embodiments 14-39, wherein R⁷ is propyl (e.g., isopropyl).

Embodiment 43 provides the method according to any one of embodiments 14-39, wherein R⁷ is difluoromethyl or trifluoromethyl.

Embodiment 44 provides the method according to embodiment 14, wherein the PRMT5 inhibitor is of the formula:

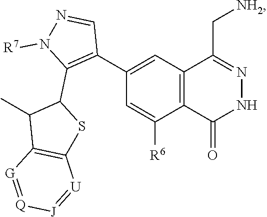

wherein
G, Q, J, and U together with the thiophene to which they are attached form:

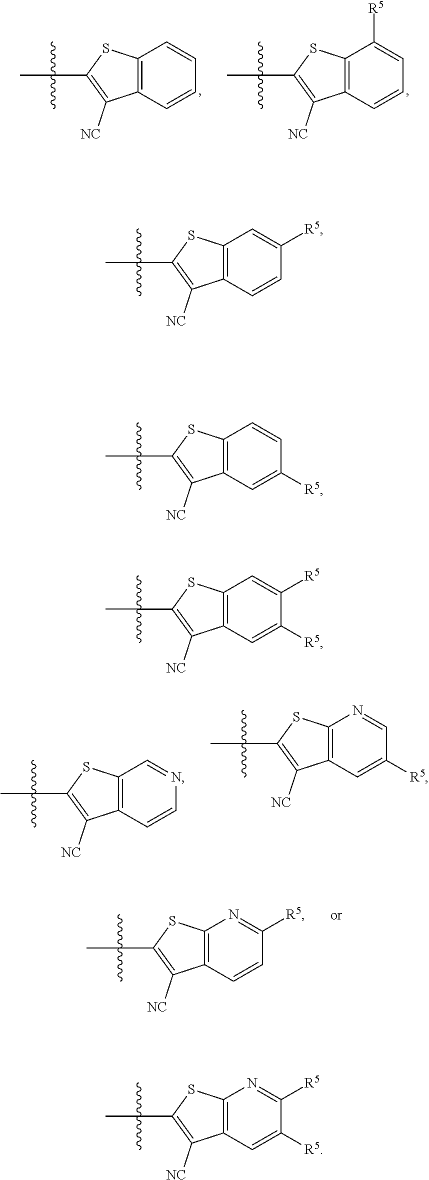

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 45 provides the method according to embodiment 14, wherein the PRMT5 inhibitor is of the formula:

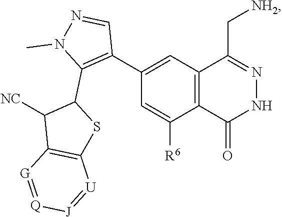

wherein
G, Q, J, and U together with the thiophene to which they are attached form:

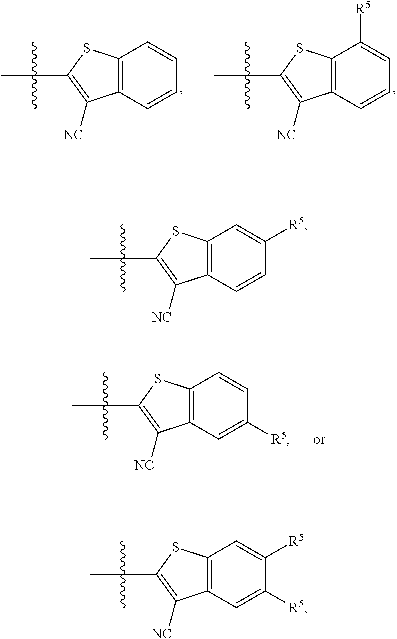

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

Embodiment 46 provides the method according to embodiment 14, wherein the PRMT5 inhibitor is of the formula:

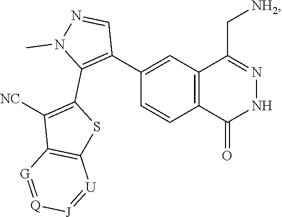

wherein
G, Q, J, and U together with the thiophene to which they are attached form:

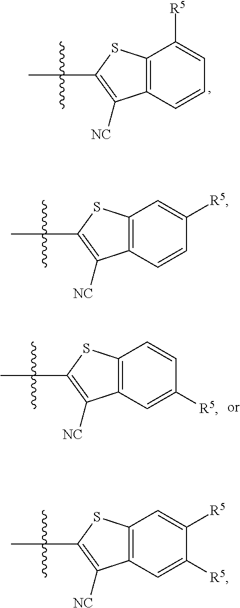

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 47 provides the method of the disclosure wherein the PRMT5 inhibitor is a compound of the formula (IIIB):

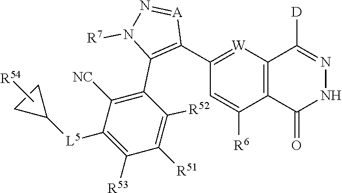

or a pharmaceutically acceptable salt thereof, wherein
A is CR⁹ or N;
D is —CH₂—NH₂,

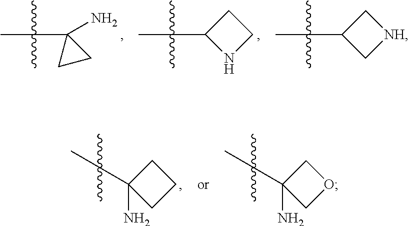 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R⁵¹ is hydrogen, fluoro, chloro, or methyl, or R⁵¹ and R⁵² together with atoms to which they are attached form a C₄-C₆ heterocycloalkyl (e.g, hydrofuranyl);
R⁵² is fluoro, chloro, or methyl, or R⁵² and R⁵³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy;
L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl.

Embodiment 48 provides the method according to embodiment 47, wherein:
A is —CH or —CCH₃;
D is —CH₂—NH₂;
W is —CH, —CCH₃, or N;
R⁵¹, R⁵², R⁵³, and R⁵⁴ are each independently selected from hydrogen, fluoro, chloro, or methyl;
L⁵ is —O—;
R⁶ is hydrogen, fluoro, chloro, or methyl; and
R⁷ is C₁-C₂ alkyl or C₁-C₂ haloalkyl.

Embodiment 49 provides the method according to embodiment 47 or embodiment 48, wherein:
A and W are —CH;
D is —CH₂—NH₂;
R⁵¹, R⁵², and R⁵³ are each independently selected from hydrogen, fluoro, chloro, and methyl;
R⁵⁴ is hydrogen;
L⁵ is —O—;
R⁶ is hydrogen; and
R⁷ is methyl.

Embodiment 50 provides the method according to any of embodiments 47-49, wherein:
A and W are —CH;
D is —CH₂—NH₂;
R⁵¹ and R⁵² are each independently selected from fluoro, chloro, and methyl;
R⁵³ and R⁵⁴ are hydrogen;
L⁵ is —O—;
R⁶ is hydrogen; and
R⁷ is methyl.

Embodiment 51 provides the method according to embodiment 47, wherein A is CH.

Embodiment 52 provides the method according to embodiment 47 or 48, wherein W is N.

Embodiment 53 provides the method according to embodiment 47 or 48, wherein W is CH.

Embodiment 54 provides the method according to any of embodiments 47-50, wherein D is —CH₂—NH₂.

Embodiment 55 provides the method according to any of embodiments 47-51, wherein R⁵⁴ is hydrogen or methyl.

Embodiment 56 provides the method according to any of embodiments 47-51, wherein R⁵⁴ is hydrogen.

Embodiment 57 provides the method according to any of embodiments 47-51, wherein R⁵⁴ is methyl.

Embodiment 58 provides the method according to embodiment 47, where the PRMT5 inhibitor is of the formula:

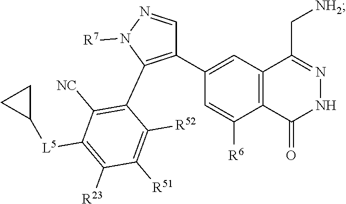

such as e.g.,

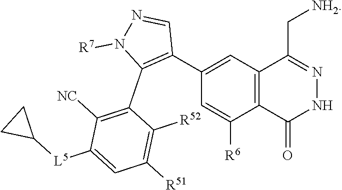

Embodiment 59 provides the method according to any of embodiments 47-55, wherein L⁵ is —CH₂—.

Embodiment 60 provides the method according to any of embodiments 47-55, wherein L⁵ is —O—.

Embodiment 61 provides the method according to any of embodiments 47-57, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 62 provides the method according to any of embodiments 47-57, wherein R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

Embodiment 63 provides the method according to any of embodiments 47-57, wherein R⁶ is hydrogen, chloro, fluoro, methyl, ethyl, methoxy, or ethoxy.

Embodiment 64 provides the method according to any of embodiments 47-57, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 65 provides the method according to any of embodiments 47-57, wherein R⁶ is halogen, C₁-C₆ alkyl, or C₁-C₆ alkoxy; for example, R⁶ is halogen, C₁-C₃ alkyl, or C₁-C₃ alkoxy.

Embodiment 66 provides the method according to any of embodiments 47-57, wherein R⁶ is chloro, fluoro, methyl, ethyl, methoxy, or ethoxy.

Embodiment 67 provides the method according to any one of embodiments 47-63, wherein R⁷ is methyl.

Embodiment 68 provides the method according to any one of embodiments 47-63, wherein R⁷ is ethyl.

Embodiment 69 provides the method according to any one of embodiments 47-63, wherein R⁷ is propyl (e.g., isopropyl).

Embodiment 70 provides the method according to any one of embodiments 47-63, wherein R⁷ is difluoromethyl or trifluoromethyl.

Embodiment 71 provides the method according to any of embodiments 47-67, wherein R⁵³ is hydrogen or methoxy; or wherein R⁵³ is hydrogen.

Embodiment 72 provides the method according to embodiment 47, where the PRMT5 inhibitor is of the formula:

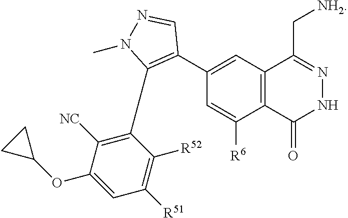

Embodiment 73 provides the method according to any one of embodiments 47-69, wherein R⁵² is fluoro, and R⁵¹ is hydrogen, fluoro, chloro, or methyl.

Embodiment 74 provides the method according to any one of embodiments 47-69, wherein R⁵² is fluoro, and R⁵¹ is chloro.

Embodiment 75 provides the method according to any one of embodiments 47-69, wherein R⁵² is fluoro, and R⁵¹ is methyl or hydrogen (for example, R⁵² is fluoro and R⁵¹ is methyl; or R⁵² is fluoro and R⁵¹ is hydrogen).

Embodiment 76 provides the method according to any one of embodiments 47-69, wherein R⁵¹ and R⁵² together with atoms to which they are attached form a hydrofuranyl (e.g.,

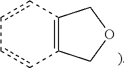

Embodiment 77 provides the method according to any one of embodiments 47-76, wherein the PRMT5 inhibitor is

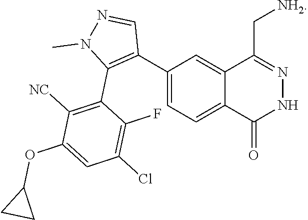 [SMILES: Cn1ncc(-c2ccc3c(=O)[nH]nc(CN)c3c2)c1-c1c(F)c(Cl)cc(OC2CC2)c1C#N]

Embodiment 78 provides the method according to any one of embodiments 47-77, wherein the PRMT5 inhibitor is

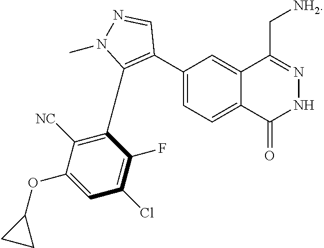

One aspect of the disclosure provides the method wherein the PRMT5 inhibitor is a compound of the formula (IIIA) (Embodiment 79):

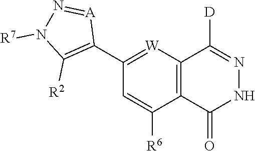

or a pharmaceutically acceptable salt thereof, wherein
A is CR⁹ or N;
D is —CH₂—NH₂,

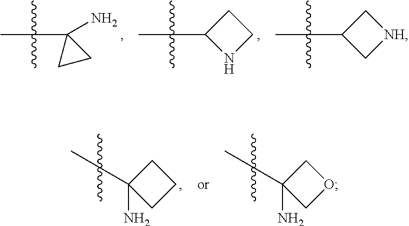 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

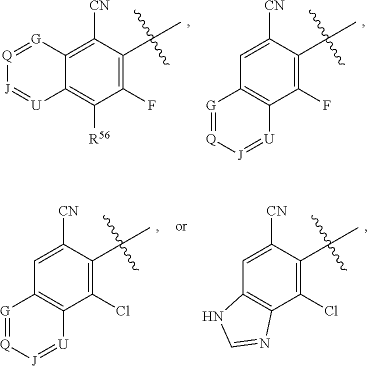

where R⁵⁶ is hydrogen, fluoro, chloro, or methyl,
G, Q, J and U are independently selected from C(H), C(R⁵), and N, provided only one or two of G, Q, J, and U can be N;
each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

One aspect of the disclosure provides the method wherein the PRMT5 inhibitor is a compound of the formula (IIIA) (Embodiment 80):

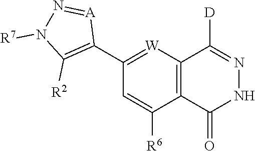

or a pharmaceutically acceptable salt thereof, wherein
A is CR⁹ or N;
D is —CH₂—NH₂,

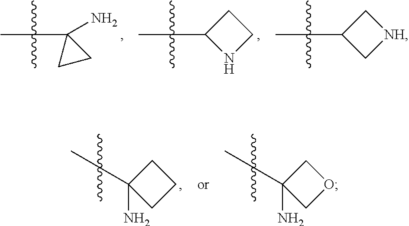 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

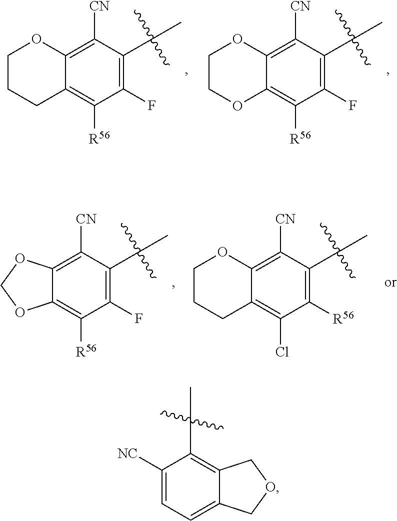

where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

Embodiment 81 provides the method according to embodiment 79 or 80, wherein A is CH.

Embodiment 82 provides the method according to embodiment 79 or 80, wherein W is N.

Embodiment 83 provides the method according to embodiment 79 or 80, wherein W is CH.

Embodiment 84 provides the method according to any of embodiments 79 or 80, wherein D is —CH₂—NH₂.

Embodiment 85 provides the method according to embodiment 79 or 80, which is of the formula:

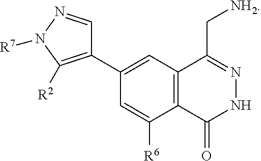

Embodiment 86 provides the method according to embodiment 79 or 81-85, wherein R² is

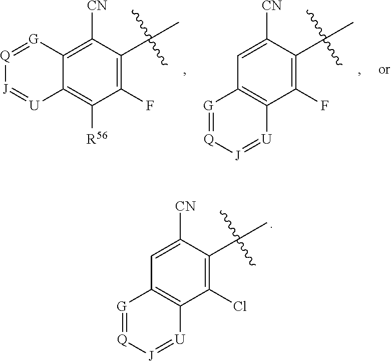

Embodiment 87 provides the method according to embodiment 86, wherein G, Q, J and U are independently selected from C(H) and C(R⁵).

Embodiment 88 provides the method according to embodiment 86, wherein G, Q, J and U are independently C(H).

Embodiment 89 provides the method according to embodiment 86, wherein at least one of G, Q, J, and U is C(R⁵), and the remaining G, Q, J, and U are independently C(H); for example only one of G, Q, J, and U is C(R⁵).

Embodiment 90 provides the method according to embodiment 86, wherein U is N, and G, Q, and J are independently selected from C(H) and C(R⁵).

Embodiment 91 provides the method according to embodiment 86, wherein G is N, and Q, J, and U are independently selected from C(H) and C(R⁵).

Embodiment 92 provides the method according to any one of embodiments 79 or 81-91, wherein R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

Embodiment 93 provides the method according to any one of embodiments 79 or 81-91, wherein R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

Embodiment 94 provides the method according to any one of embodiments 79 or 81-91, wherein R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

Embodiment 95 provides the method according to any one of embodiments 79 or 81-91, wherein R⁵, if present, is halogen, C₁-C₆ alkyl, or C₁-C₆ alkoxy; for example, R⁶ is halogen, C₁-C₃ alkyl, or C₁-C₃ alkoxy.

Embodiment 96 provides the method according to any one of embodiments 79 or 81-91, wherein R⁵, if present, is chloro, fluoro, methyl, ethyl, methoxy, or ethoxy.

Embodiment 97 provides the method according to any one of embodiments 79 or 81-91, wherein R⁵⁶ is fluoro, chloro, or methyl.

Embodiment 98 provides the method according to embodiment 80-85, wherein R² is

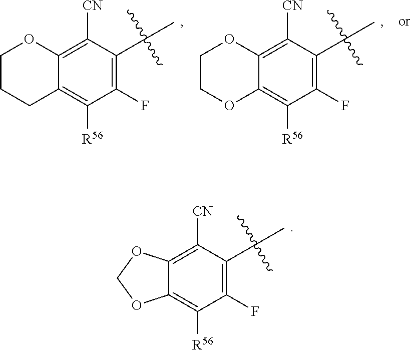

Embodiment 99 provides the method according to any of embodiments 80-85 or 98, wherein R⁵⁶ is hydrogen, fluoro, chloro, or methyl.

Embodiment 100 provides the method according to any of embodiments 79-99, wherein R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 101 provides the method according to any of embodiments 79-99, wherein R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

Embodiment 102 provides the method according to any of embodiments 79-99, wherein R⁶ is hydrogen, chloro, fluoro, methyl, ethyl, methoxy, or ethoxy.

Embodiment 103 provides the method according to any of embodiments 79-99, wherein R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

Embodiment 104 provides the method according to any of embodiments 79-99, wherein R⁶ is halogen, C₁-C₆ alkyl, or C₁-C₆ alkoxy; for example, R⁶ is halogen, C₁-C₃ alkyl, or C₁-C₃ alkoxy.

Embodiment 105 provides the method according to any of embodiments 79-99, wherein R⁶ is chloro, fluoro, methyl, ethyl, methoxy, or ethoxy.

Embodiment 106 provides the method according to any one of embodiments 79-105, wherein R⁷ is methyl.

Embodiment 107 provides the method according to any one of embodiments 79-105, wherein R⁷ is ethyl.

Embodiment 108 provides the method according to any one of embodiments 79-105, wherein R⁷ is propyl (e.g., isopropyl).

Embodiment 109 provides the method according to any one of embodiments 79-105, wherein R⁷ is difluoromethyl or trifluoromethyl.

In certain embodiments of the methods of the disclosure as described herein, the PRMT5 inhibitor is:

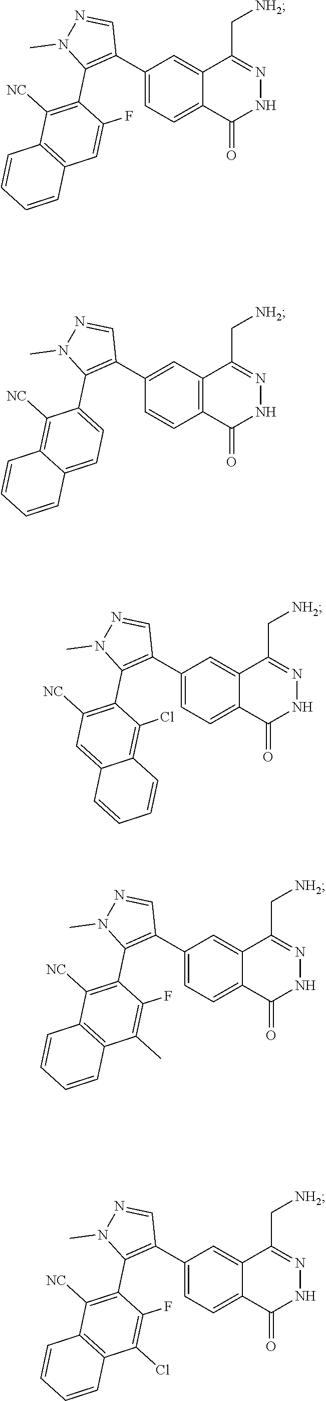 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N ; Cn1ncc(-c2ccc3c(=O)[nH]nc(CN)c3c2)c1-c1c(C#N)cc2ccccc2c1Cl ; Cc1c(F)c(-c2c(-c3ccc4c(=O)[nH]nc(CN)c4c3)cnn2C)c(C#N)c2ccccc12 ; Cn1ncc(-c2ccc3c(=O)[nH]nc(CN)c3c2)c1-c1c(F)c(Cl)c2ccccc2c1C#N]

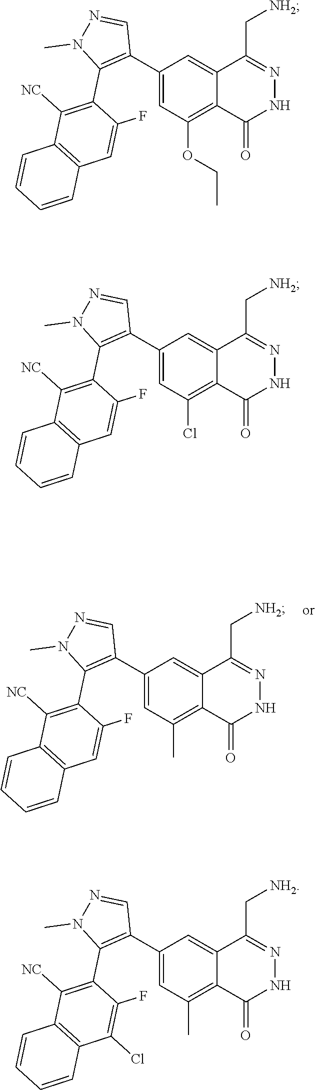 [SMILES: CCOc1cc(-c2cnn(C)c2-c2c(F)cc3ccccc3c2C#N)cc2c(CN)n[nH]c(=O)c12 ; Cn1ncc(-c2cc(Cl)c3c(=O)[nH]nc(CN)c3c2)c1-c1c(F)cc2ccccc2c1C#N ; Cc1cc(-c2cnn(C)c2-c2c(F)cc3ccccc3c2C#N)cc2c(CN)n[nH]c(=O)c12 ; Cc1cc(-c2cnn(C)c2-c2c(F)c(Cl)c3ccccc3c2C#N)cc2c(CN)n[nH]c(=O)c12]

In certain embodiments of the methods of the disclosure as described herein, the PRMT5 inhibitor is:

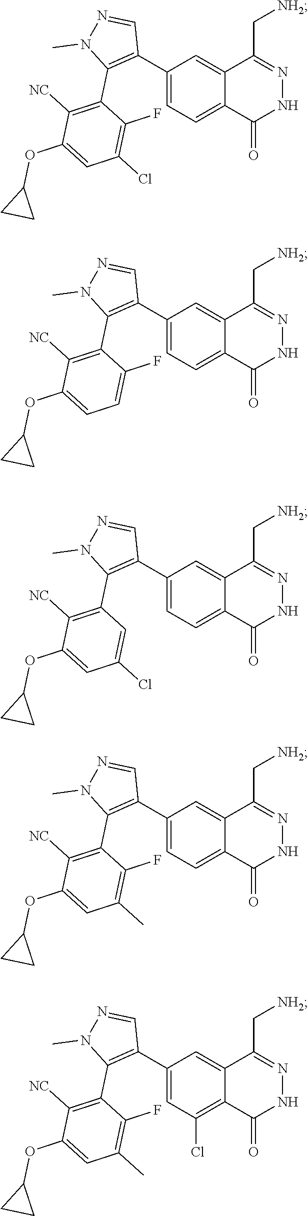 [SMILES: Cn1ncc(-c2ccc3c(=O)[nH]nc(CN)c3c2)c1-c1c(F)c(Cl)cc(OC2CC2)c1C#N ; Cn1ncc(-c2ccc3c(=O)[nH]nc(CN)c3c2)c1-c1c(F)ccc(OC2CC2)c1C#N ; Cn1ncc(-c2ccc3c(=O)[nH]nc(CN)c3c2)c1-c1cc(Cl)cc(OC2CC2)c1C#N ; Cc1cc(OC2CC2)c(C#N)c(-c2c(-c3ccc4c(=O)[nH]nc(CN)c4c3)cnn2C)c1F ; Cc1cc(OC2CC2)c(C#N)c(-c2c(-c3cc(Cl)c4c(=O)[nH]nc(CN)c4c3)cnn2C)c1F]

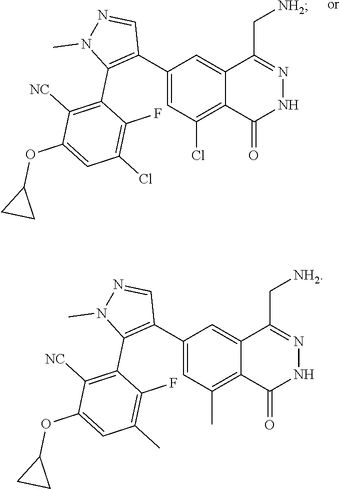 [SMILES: Cn1ncc(-c2cc(Cl)c3c(=O)[nH]nc(CN)c3c2)c1-c1c(F)c(Cl)cc(OC2CC2)c1C#N ; "or" ; Cc1cc(OC2CC2)c(C#N)c(-c2c(-c3cc(C)c4c(=O)[nH]nc(CN)c4c3)cnn2C)c1F]

In certain embodiments of the methods of the disclosure as described herein, the PRMT5 inhibitor is:

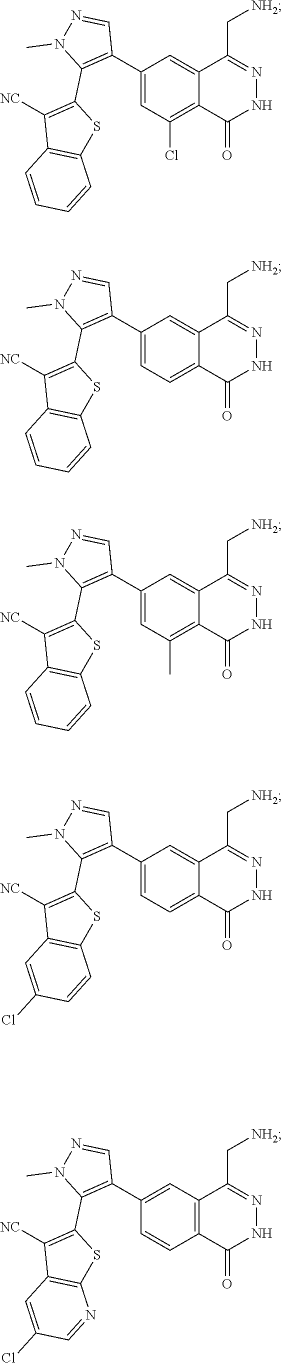 [SMILES: Cn1ncc(-c2cc(Cl)c3c(=O)[nH]nc(CN)c3c2)c1-c1sc2ccccc2c1C#N ; Cn1ncc(-c2ccc3c(=O)[nH]nc(CN)c3c2)c1-c1sc2ccccc2c1C#N ; Cc1cc(-c2cnn(C)c2-c2sc3ccccc3c2C#N)cc2c(CN)n[nH]c(=O)c12 ; Cn1ncc(-c2ccc3c(=O)[nH]nc(CN)c3c2)c1-c1sc2ccc(Cl)cc2c1C#N ; Cn1ncc(-c2ccc3c(=O)[nH]nc(CN)c3c2)c1-c1sc2ncc(Cl)cc2c1C#N]

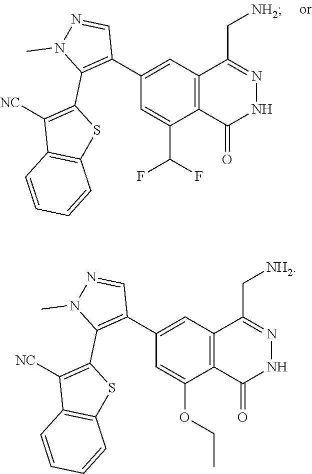 [SMILES: Cn1ncc(-c2cc(C(F)F)c3c(=O)[nH]nc(CN)c3c2)c1-c1sc2ccccc2c1C#N ; CCOc1cc(-c2cnn(C)c2-c2sc3ccccc3c2C#N)cc2c(CN)n[nH]c(=O)c12]

In certain embodiments of the methods of the disclosure as described herein, the PRMT5 inhibitor is:

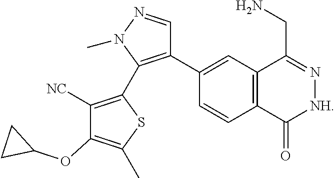 [SMILES: Cc1sc(-c2c(-c3ccc4c(=O)[nH]nc(CN)c4c3)cnn2C)c(C#N)c1OC1CC1]

In an aspect, the present disclosure provides for a method for treating cancer in a subject, the method comprising administering to the subject:
a therapeutically effective amount of ABT-199 (venetoclax), wherein ABT-199 is:

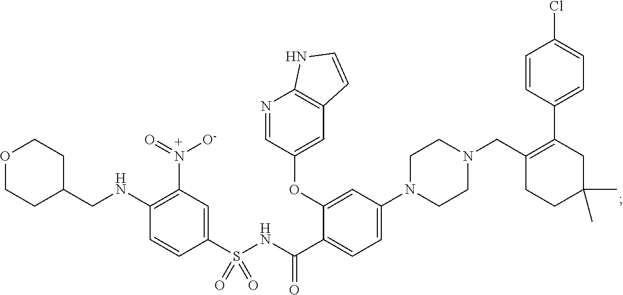

and
   a therapeutically effective amount of a PRMT5 inhibitor of formula:

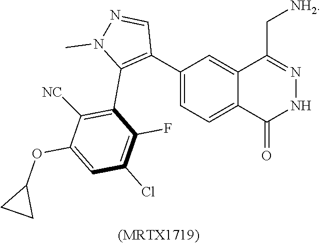

In an aspect, the present disclosure provides for a method for treating cancer in a subject, the method comprising administering to the subject:
a therapeutically effective amount of ABT-263 (navitoclax), wherein ABT-263 is:

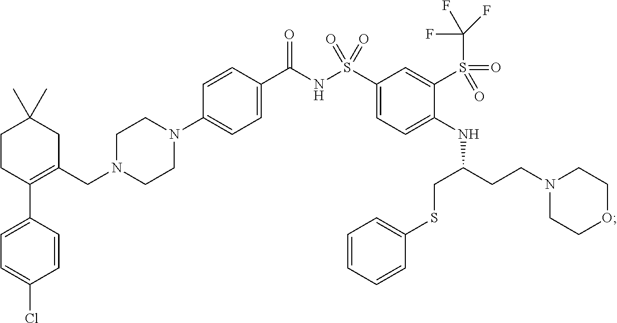 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

and
   a therapeutically effective amount of a PRMT5 inhibitor of formula:

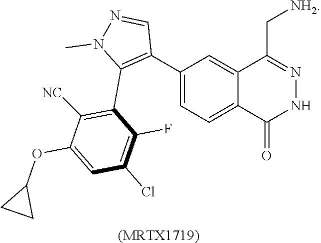

The PRMT5 inhibitor of the disclosure and/or the BCL-2 family inhibitor (e.g., ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax and/or DT2216) of the disclosure may be provided as a pharmaceutical composition comprising a therapeutically effective amount of such inhibitor and a pharmaceutically acceptable carrier, excipient, and/or diluents. The PRMT5 inhibitor of the disclosure and/or the BCL-2 family inhibitor of the disclosure may be formulated by any method well known in the art and may be prepared for administration by any route, including, without limitation, parenteral, oral, sublingual, transdermal, topical, intranasal, intratracheal, or intrarectal.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions of the disclosure may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

The PRMT5 inhibitor and BCL-2 family inhibitor of the disclosure are administered in a therapeutically effective amount. As used herein, the phrase “therapeutically effective amount” or “effective amount” refers to the amount of active agent that elicits the biological or medicinal response that is being sought in a tissue, system, subject or human by a researcher, medical doctor or other clinician. In general, the therapeutically effective amount is sufficient to deliver the biological or medicinal response to the subject without causing serious toxic effects. A dose of the active agent may be in the range from about 1 to 500 mg/m² per day, such as 5 to 400 mg/m² per day, more generally 10 to 300 mg/m² body weight of the recipient per day. A typical topical dosage will range from 0.01 to 10% wt/wt in a suitable carrier.

In certain embodiments of the methods of the disclosure, the therapeutically effective amount of the PRMT5 inhibitor is in the range of about 0.01 to 300 mg/kg per day. For example, in certain embodiments, the therapeutically effective amount of the PRMT5 inhibitor is in the range of about 0.1 to 100 mg/kg per day, or 25 to 100 mg/kg per day, or 50 to 100 mg/kg per day.

In certain embodiments, the therapeutically effective amount of the PRMT5 inhibitor is less than 1% of, e.g., less than 10%, or less than 25%, or less than 50% of the clinically-established therapeutic amount (e.g., such as the amount required when the PRMT5 inhibitor is administered by itself).

In certain embodiments of the methods of the disclosure, the therapeutically effective amount of the BCL-2 family inhibitor is in the range of about 1 to 500 mg/m² per day, such as 5 to 400 mg/m² per day, more generally 10 to 300 mg/m² body weight of the recipient per day. For example, in certain embodiments, the therapeutically effective amount of the BCL-2 family inhibitor is in the range of about 30 to 300 mg/m² per day (e.g., 50 to 250 mg/m², or 50 to 200 mg/m², or 50 to 150 mg/m² per day).

For example, in various embodiments, the BCL-2 family inhibitor may be ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax or DT2216. Accordingly, in certain embodiments of the methods of the disclosure, the therapeutically effective amount of each of these agents is in the range of about 1 to 500 mg/m² per day, such as 5 to 400 mg/m² per day, more generally 10 to 300 mg/m² body weight of the recipient per day. For example, in certain embodiments, the therapeutically effective amount of any one of these agents is in the range of about 30 to 300 mg/m² per day (e.g., 50 to 250 mg/m², or 50 to 200 mg/m², or 50 to 150 mg/m² per day).

In certain embodiments, the therapeutically effective amount of ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax or DT2216 is less than 1% of, e.g., less than 10%, or less than 25%, or less than 50%, or less than 75% of the clinically-established therapeutic amount (e.g., such as the amount required when said compound is administered by itself).

Combination therapy, in defining use of PRMT5 inhibitor and the BCL-2 family inhibitor (e.g., ABT-199 (venetoclax), ABT-263 (navitoclax), A-1155463, A-1331852, obatoclax (GX15-070), ABT-737, TW-37, gossypol or (R)-(−)-gossypol, HA14-1, sabutoclax or DT2216) of the present disclosure, is intended to embrace administration of each agent in a sequential manner in a regimen that will provide beneficial effects of the drug combination (e.g., the PRMT5 inhibitor and the BCL-2 family inhibitor of the disclosure can be formulated as separate compositions that are given sequentially), and is intended as well to embrace co-administration of these agents in a substantially simultaneous manner, such as in a single dosage form having a fixed ratio of these active agents or in multiple or a separate dosage forms for each agent. The disclosure is not limited in the sequence of administration: the PRMT5 inhibitor of the disclosure may be administered either prior to or after (i.e., sequentially), or at the same time (i.e., simultaneously) as administration of the BCL-2 family inhibitor of the disclosure.

The methods of disclosure are useful as a first-line treatment. Thus, in certain embodiments of the methods of the disclosure, the subject has not previously received another first-line of therapy.

The methods of disclosure are also useful as a first-line maintenance or a second-line treatment. Thus, in certain embodiments of the methods of the disclosure, the subject has previously completed another first-line of therapy. For example, the methods of the disclosure, in certain embodiments, may provide a delay in progression and relapse of cancer in subjects that have previously completed another first-line chemotherapy. For example, in certain embodiments, the subject has previously completed therapeutic regimens that may include but are not limited to chemotherapies, targeted therapies and immunotherapies, either as single agents or in combination with other therapies. In certain embodiments of the methods of the disclosure, the subject has previously completed another first-line chemotherapy and is in partial response to such chemotherapy.

### DEFINITIONS

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which a divalent moiety is required and is stated as being “aryl,” those skilled in the art will understand that the term “aryl” refers to the corresponding divalent moiety, arylene. All atoms are understood to have their normal number of valences for bond formation (i.e., 4 for carbon, 3 for N, 2 for O, and 2, 4, or 6 for S, depending on the oxidation state of the S).

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC₁-C₆ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NRx, wherein Rx is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C₁-C₆)alkyl(C₆-C₁₀) aryl, including, without limitation, benzyl, phenethyl, and naphthylmethyl. For example, an arC₁-C₃alkyl is an aryl group covalently linked to a C₁-C₃ alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently-C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via an alkylene linker.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. Heteroaryl also includes fused multicyclic (e.g., bicyclic) ring systems in which one or more of the fused rings is non-aromatic, provided that at least one ring is aromatic and at least one ring contains an N, O, or S ring atom. Examples of heteroaryl groups include acridinyl, azocinyl, benzimidazolyl, benzofuranyl, benzo[d]oxazol-2 (3H)-one, 2H-benzo[b][1,4]oxazin-3 (4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via an alkylene linker. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, fluorochloromethyl, chloromethyl, and fluoromethyl.

The term “hydroxyalkyl” refers to -alkylene-OH.

### EXAMPLES

The methods of the disclosure are illustrated further by the following examples, which is not to be construed as limiting the disclosure in scope or spirit to the specific procedures and compounds described in them.

### Example 1—In Vivo Combination Study (LU99 Model)

Immunodeficient female nu/nu mice were implanted with 5×10⁶ LU99 cancer cells in 50% Matrigel. Tumor volume measurements were collected using calipers and determined utilizing the formula 0.5×L×W² in which L refers to length and W refers to width of each tumor. When tumors reached approximately 125-150 mm³, animals were randomized to receive A) vehicle, B) a PRMT5 inhibitor, C) navitoclax (ABT-263), D) venetoclax (ABT-199), E) A-1331852, F) the PRMT5 inhibitor and navitoclax, F) the PRMT5 inhibitor and venetoclax, or G) the PRMT5 inhibitor and A-1331852 all administered orally (PO) with the indicated dose and schedule for 20-34 days. Tumor volume was measured twice a week (n=5/treatment group). Average tumor volume and standard error of the mean was calculated and plotted at each study day using GraphPad Prism.

The PRMT5 inhibitor was MRTX1719 administered at 100 mg/kg once a day (QD). MRTX1719 is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, disclosed as Example 16-8 at p. 307 of the international patent publication No. WO 2021/050915 A1, published 18 Mar. 2021, incorporated by reference in its entirety.

The BCL-2 family inhibitors used in this example, ABT-199 (venetoclax) and ABT-263 (navitoclax), were administered at 100 mg/kg once a day (QD) and supplied by Selleck Chemicals (Houston, TX).

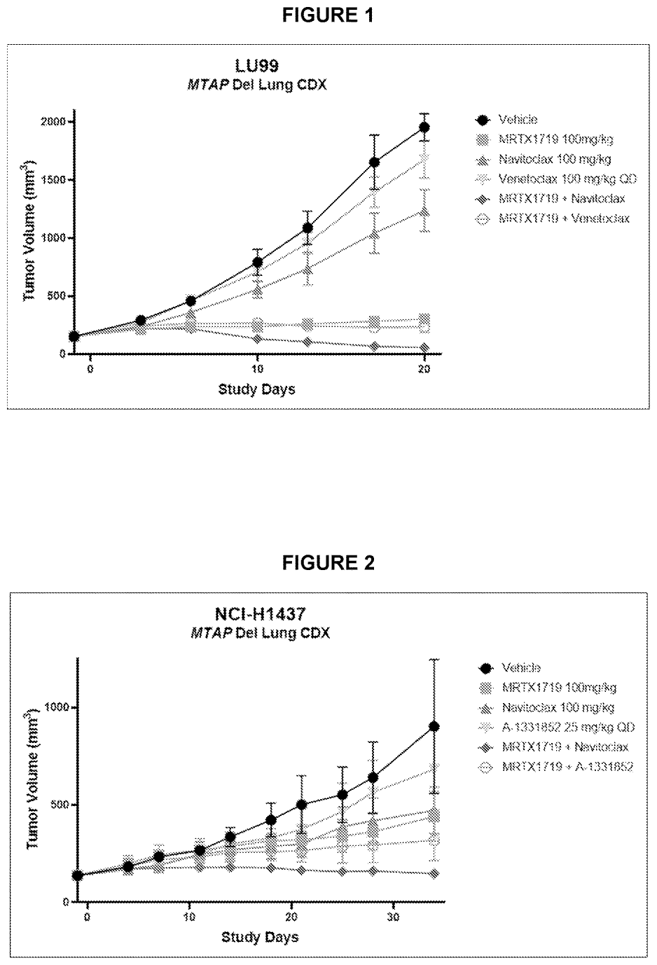

Results are provided in FIG. 1 and Table 1.

TABLE 1
Average Tumor Volume (mm\hat{ }3)

| | | | ABT- | ABT- | MRTX1719 + | MRTX1719 + |
|---|---|---|---|---|---|---|
| Day | Vehicle | MRTX1719 | 263 | 199 | ABT-263 | ABT-199 |
| −1 | 153 | 153 | 153 | 153 | 153 | 153 |
| 3 | 292 | 215 | 236 | 270 | 221 | 242 |
| 6 | 457 | 236 | 359 | 462 | 219 | 265 |
| 10 | 792 | 236 | 558 | 709 | 132 | 267 |
| 13 | 1088 | 260 | 737 | 956 | 107 | 242 |
| 17 | 1653 | 282 | 1042 | 1395 | 67 | 232 |
| 20 | 1820 | 302 | 1237 | 1675 | 56 | 236 |

The combination of MRTX1719 with ABT-199 (venetoclax) and ABT-263 (navitoclax), respectively, lead to greater antitumor activity, as measured by change in tumor volume over time, compared to either compound alone in the LU99 cell line derived xenograft model.

### Example 2—In Vivo Combination Study (NCI-H1437 Model)

Example 2 was carried out using NCI-H1437 cell line derived xenografts according to the in vivo study procedure described above in Example 1.

The PRMT5 inhibitor was MRTX1719 administered at 100 mg/kg once a day (QD). MRTX1719 is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, disclosed as Example 16-8 at p. 307 of the international patent publication No. WO 2021/050915 A1, published 18 Mar. 2021, incorporated by reference in its entirety. The BCL-2 family inhibitor, ABT-263 (navitoclax), was administered at 100 mg/kg once a day (QD) and supplied by Selleck Chemicals (Houston, TX). The BCL-2 family inhibitor A-1331852 was administered at 25 mg/kg once a day (QD) and synthesized at Wuxi AppTec (Wuhan, China).

Results are provided in FIG. 2 and Table 2.

TABLE 2
Average Tumor Volume (mm\hat{ }3)

| | | | ABT- | A- | MRTX1719 + | MRTX1719 + |
|---|---|---|---|---|---|---|
| Day | Vehicle | MRTX1719 | 263 | 852 | ABT-263 | A-852 |
| −1 | 138 | 139 | 138 | 139 | 137 | 138 |
| 4 | 184 | 200 | 172 | 171 | 172 | 186 |
| 7 | 234 | 244 | 192 | 191 | 178 | 219 |
| 11 | 267 | 268 | 244 | 248 | 179 | 234 |
| 14 | 336 | 288 | 269 | 300 | 180 | 256 |
| 18 | 423 | 316 | 288 | 332 | 178 | 260 |
| 21 | 502 | 322 | 299 | 376 | 166 | 265 |
| 25 | 553 | 340 | 389 | 467 | 159 | 289 |
| 28 | 640 | 362 | 420 | 564 | 162 | 294 |
| 34 | 903 | 441 | 472 | 683 | 148 | 319 |

The combination of MRTX1719 with ABT-263 (navitoclax) and A-1331852 lead to greater antitumor activity, as measured by change in tumor volume over time, compared to either compound alone in the NCI-H1437 cell line derived xenograft model.

### Example 3—In Vivo Combination Study (SW1573 Model)

Example 3 was carried out in SW1573 cell line derived xenografts according to the in vivo study procedure described above in Example 1.

The PRMT5 inhibitor was MRTX1719 administered at 100 mg/kg once a day (QD). MRTX1719 is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, disclosed as Example 16-8 at p. 307 of the international patent publication No. WO 2021/050915 A1, published 18 Mar. 2021, incorporated by reference in its entirety. The BCL-2 family inhibitor, ABT-263 (navitoclax), was administered at 100 mg/kg once a day (QD) and supplied by Selleck Chemicals (Houston, TX). The BCL-2 family inhibitor A-1331852 was administered at 25 mg/kg once a day (QD) and synthesized at Wuxi AppTec (Wuhan, China).

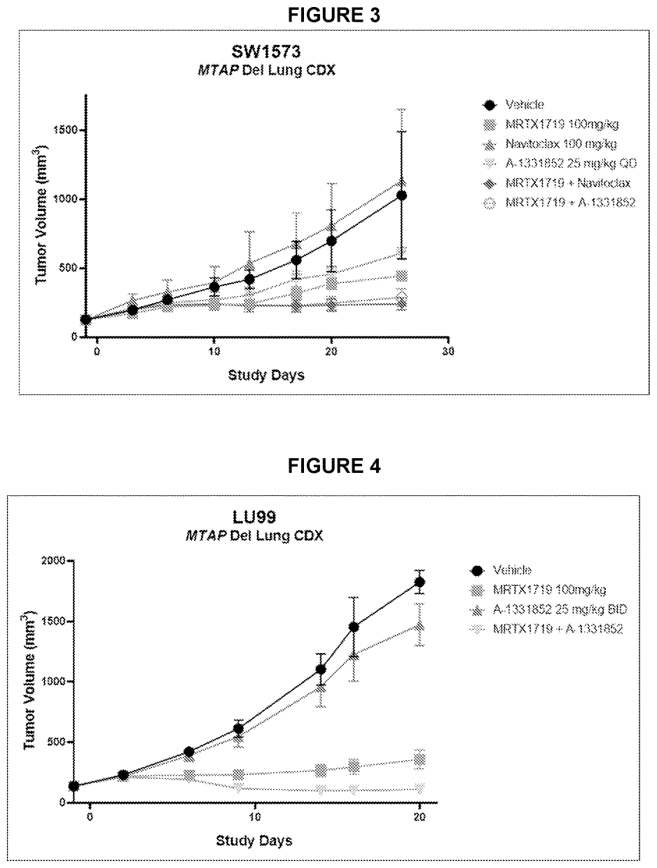

Results are provided in FIG. 3 and Table 3

TABLE 3
Average Tumor Volume (mm\hat{ }3)

| | | | ABT- | A- | MRTX1719 + | MRTX1719 + |
|---|---|---|---|---|---|---|
| Day | Vehicle | MRTX1719 | 263 | 852 | ABT-263 | A-852 |
| −1 | 126 | 126 | 123 | 125 | 127 | 123 |
| 3 | 196 | 172 | 265 | 209 | 209 | 193 |
| 6 | 272 | 220 | 328 | 250 | 232 | 237 |
| 10 | 363 | 229 | 395 | 271 | 240 | 232 |
| 13 | 419 | 242 | 536 | 305 | 228 | 235 |
| 17 | 559 | 319 | 678 | 421 | 228 | 227 |
| 20 | 697 | 387 | 809 | 456 | 233 | 248 |
| 26 | 1027 | 444 | 1133 | 609 | 241 | 288 |

The combination of MRTX1719 with ABT-263 (navitoclax) and A-1331852 lead to greater antitumor activity, as measured by change in tumor volume over time, compared to either compound alone in the SW173 cell line derived xenograft model.

### Example 4—In Vivo Combination Study (LU99 Model)

Example 4 was carried out according to the in vivo study procedure described above in Example 1.

The PRMT5 inhibitor was MRTX1719 administered at 100 mg/kg once a day (QD). MRTX1719 is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, disclosed as Example 16-8 at p. 307 of the international patent publication No. WO 2021/050915 A1, published 18 Mar. 2021, incorporated by reference in its entirety. The BCL-2 family inhibitor, A-1331852 was administered at 25 mg/kg twice a day (BID) and synthesized at Wuxi AppTec (Wuhan, China).

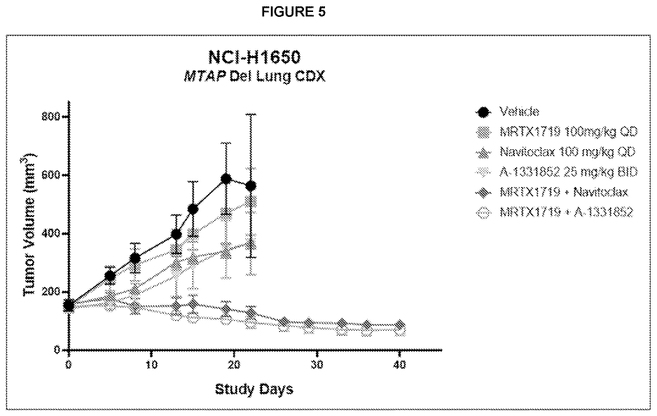

Results are provided in FIG. 4 and Table 4.

TABLE 4
Average Tumor Volume (mm\hat{ }3)

| Day | Vehicle | MRTX1719 | A-852 | MRTX1719 + A-852 |
|---|---|---|---|---|
| −1 | 137 | 137 | 137 | 136 |
| 2 | 231 | 221 | 217 | 213 |
| 6 | 422 | 229 | 393 | 193 |
| 9 | 614 | 232 | 547 | 118 |
| 14 | 1103 | 268 | 960 | 98 |
| 16 | 1455 | 296 | 1228 | 101 |
| 20 | 1683 | 360 | 1331 | 110 |

The combination of MRTX1719 with A-1331852 lead to greater antitumor activity, as measured by change in tumor volume over time, compared to either compound alone in the LU99 cell line derived xenograft model.

### Example 5—In Vivo Combination Study (NCI-H1650 Model)

Example 5 was carried out using NCI-H1650 cell line derived xenografts according to the in vivo study procedure described above in Example 1.

The PRMT5 inhibitor was MRTX1719 administered at 100 mg/kg once a day (QD). MRTX1719 is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, disclosed as Example 16-8 at p. 307 of the international patent publication No. WO 2021/050915 A1, published 18 Mar. 2021, incorporated by reference in its entirety. The BCL-2 family inhibitor, ABT-263 (navitoclax), was administered at 100 mg/kg once a day (QD) and supplied by Selleck Chemicals (Houston, TX). The BCL-2 family inhibitor A-1331852 was administered at 25 mg/kg twice a day (BID) and synthesized at Wuxi AppTec (Wuhan, China).

Results are provided in FIG. 5 and Table 5.

TABLE 5
Average Tumor Volume (mm\hat{ }3)

| | | | ABT- | A- | MRTX1719 + | MRTX1719 + |
|---|---|---|---|---|---|---|
| Day | Vehicle | MRTX1719 | 263 | 852 | ABT-263 | A-852 |
| 0 | 154 | 149 | 155 | 143 | 160 | 151 |
| 5 | 255 | 246 | 186 | 163 | 177 | 153 |
| 8 | 316 | 292 | 210 | 189 | 151 | 147 |
| 13 | 398 | 344 | 302 | 255 | 152 | 120 |
| 15 | 484 | 398 | 319 | 291 | 158 | 112 |
| 19 | 588 | 469 | 339 | 347 | 141 | 106 |
| 22 | 564 | 510 | 371 | 366 | 128 | 95 |
| 26 | | | | | 98 | 84 |
| 29 | | | | | 94 | 78 |
| 33 | | | | | 92 | 71 |
| 36 | | | | | 87 | 69 |
| 40 | | | | | 87 | 70 |

The combination of MRTX1719 with ABT-263 (navitoclax) and A-1331852 lead to greater antitumor activity, as measured by change in tumor volume over time, compared to either compound alone in the NCI-H1650 cell line derived xenograft model.

### Example 6—Synergy Scores, In Vitro Combination Study

This Example illustrates that the combination of exemplary PRMT5 inhibitor compounds of the type described in WO2021/050915 and BCL-2 family inhibitors synergistically inhibits the growth of MTAP-deleted cancer cell lines.

A panel of MTAP-deleted cancer cell lines was assembled to determine whether combining BCL-2 family inhibitors with exemplary PRMT5 inhibitors disclosed herein results in synergistic activity.

[Assays for determining the synergy score for the pairwise combinations for each cell line were performed in triplicate. 384 or 96-well plates plus additional wells of a separate 384 or 96-well control plate for determining baseline luminescence were seeded with cells of a particular MTAP-deleted cell line in a suitable growth medium for that cell line, e.g., RPMI 1640 medium supplemented with 10% FBS and any cell line specific reagents needed for growth. The plates were incubated overnight at 37 °C in a 5% CO₂ atmosphere.

To each of the designated baseline wells, Cell-Titer Glo reagent (CTG; Promega Corporation) was added to each well and the plates were incubated for 20 min shaking at room temperature. Baseline luminescence was quantitated using a BMG ClarioStar multimode plate reader according to the manufacturer's instructions.

A series of 1000× drug dilutions in 100% DMSO was prepared that includes a 9-point single agent 3-fold dilution of the exemplary PRMT5 inhibitor (of the type described in WO2021/050915) with a top dose of 3000 nM (and a 6-point single agent 5-fold dilution of the BCL-2 family inhibitor with a top dose of 3000 nM as reference standards.

A 10× intermediate dosing plate was prepared in serum free medium that contains arrayed single agent dilutions of exemplary PRMT5 inhibitor (of the type described in WO2021/050915) and or the BCL-2 family inhibitor. In addition, a matrix of 54 dilution combinations of exemplary PRMT5 inhibitor (of the type described in WO2021/050915) and the BCL-2 family inhibitor was prepared as test samples.

To each corresponding well of the 384-well plates seeded with the appropriate cell line above, 10× single agent and the 54 combinations of the dose matrix was added and the plates were incubated for 120 hours at 37 °C in 5% CO₂ atmosphere. Cell-Titer Glo reagent (CTG) was added to each test well, the plates were incubated for 20 min shaking at room temperature, and luminescence was quantitated using a BMG ClarioStar multimode plate reader according to the manufacturer's instructions. To normalize test samples, the determined baseline luminescence was subtracted from each test sample prior to analysis and mean values are calculated from replicate samples.

The raw data and generated metadata were used as input files to calculate percent effect for each treatment condition and analyzed using four independent mathematical reference models designed to determine whether the two test compounds demonstrate synergy: Loewe additivity, Bliss independence, Highest Single Agent and ZIP (reference needed?).

The output of the data from each mathematical model is the assignment of a relative synergy score. The data reported in Table 6 are the aggregate sum of the Loewe additivity, Bliss independence, Highest Single Agent and ZIP synergy scores (“Composite Synergy Score”).

TABLE 6
Composite Synergy Scores for Exemplary BCL-2 Family
Inhibitors Combined with Exemplary PRMT5 Inhibitors
Against MTAP-deleted Cancer Cell Lines

| | BCL-2 | BCL-2/-xL/-W | BCL-xL | |
|---|---|---|---|---|
| Cell Line | Venetoclax | Navitoclax | A-1155463 | A-1331852 |
| A549 | −7 | 23 | 22 | 42 |
| H1437 | 2 | 13 | 17 | 36 |
| H1650 | −11 | 32 | 51 | 62 |
| H2228 | −13 | 24 | 46 | 36 |
| HCC4006 | −6 | 23 | 88 | 63 |
| LU99 | −8 | 41 | 27 | 64 |
| SW1573 | −16 | 14 | 29 | 50 |
| H2052 | −33 | 11 | 14 | 12 |
| H2452 | 28 | −20 | 37 | 42 |
| Mero14 | −8 | 18 | 12 | 28 |
| Mero83 | 20 | −26 | −11 | 11 |
| Mero84 | −12 | −4 | 0 | 0 |
| Miapaca2 | −7 | 16 | 44 | 70 |
| SU8686 | 4 | 12 | 50 | 50 |
| RT4 | 0 | 24 | 37 | 40 |

These results demonstrate that a synergistic effect was observed for the combination of a variety of BCL-2 family inhibitors with exemplary PRMT5 inhibitor compounds (of the type described in WO2021/050915) in each cell line thereby increasing the sensitivity of the MTAP-deleted cell line to the PRMT5 inhibitor.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method for treating cancer in a subject, the method comprising:
administering to the subject a therapeutically effective amount of a BCL-2 family inhibitor and a therapeutically effective amount of a protein arginine N-methyl transferase 5 (PRMT5) inhibitor.

2. The method of claim 1, wherein the cancer is associated with a methylthioadenosine phosphorylase (MTAP) gene homozygous deletion.

3. The method of claim 1 or 2, wherein the PRMT5 inhibitor is a methylthioadenosine (MTA)-cooperative PRMT5 inhibitor.

4. The method of claim 1 or 2, wherein the cancer is lung cancer, non-small cell lung cancer, pancreatic cancer, colon cancer, bladder cancer, head and neck cancer, bladder cancer, esophageal cancer, diffuse large B cell lymphoma, lymphoma, stomach cancer, melanoma, breast cancer, brain cancer, cholangiocarcinoma, mesothelioma or malignant peripheral nerve sheath tumors.

5. The methos of claim 3, wherein the cancer is mesothelioma.

6. The method of claim 3 or 4, wherein the cancer is non-small cell lung cancer.

7. The method of any of claims 1 to 6, wherein the PRMT5 inhibitor is compound of Formula IIA, IIB or IIC: 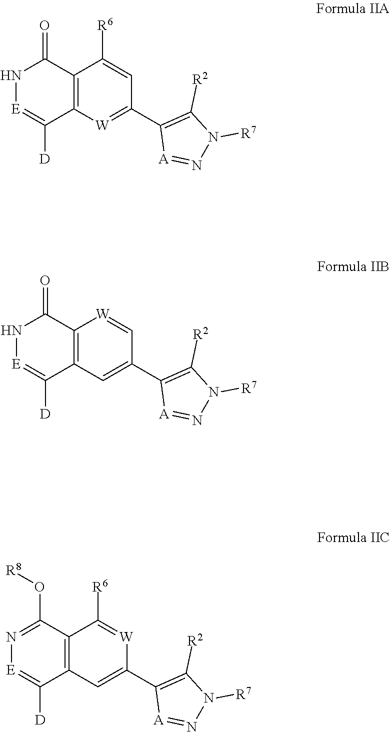
or a pharmaceutically acceptable salt thereof, wherein:
A is CR⁹ or N;
D is (C(R⁹)₂)₁₋₂—NH₂, 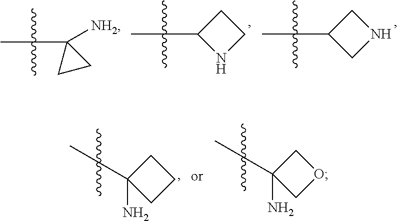
or D is 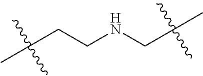 [SMILES: *CCNC*]
where the methylene is bonded to E where E is C;
E is C, CR⁹ or N;
each L is independently a bond or C₁-C₃ alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C₁-C₃alkyl, —X—(C₁-C₃ alkyl)-aryl, heteroalkyl, C₂-C₄ alkynyl, —X-haloalkyl, —X—C₁-C₅ alkyl, -Z-C₁-C₅ alkyl, heterocyclyl, —X-L-cycloalkyl, -Z-cycloalkyl, —X-aryl, -Z-aryl, or —X-heteroaryl, wherein the heterocyclyl, the cycloalkyl, the aryl and the heteroaryl are optionally substituted with one or more R⁵;
each R⁴ is independently hydrogen or C₁-C₃ alkyl;
each R⁵ is independently cyano, oxo, halogen, C₁-C₃ alkyl, hydroxyalkyl, hydroxy, alkoxy, alkoxy-C₁-C₃ alkyl, —X-haloalkyl, -Z-cycloalkyl, X—(C₁-C₃ alkyl)-aryl, X—(C₁-C₃ alkyl)-aryl substituted with cyano, —X-L-cycloalkyl optionally substituted with C₁-C₃ alkyl or oxo, —X-L-heteroaryl optionally substituted with one or more C₁-C₃ alkyl or oxo, —X-L-heterocyclyl optionally substituted with one or more C₁-C₃ alkyl or oxo, or —X-aryl;
R⁶ is hydrogen, halogen, C₁-C₃ alkyl, haloalkyl, hydroxy, alkoxy, C₁-C₃ alkyl-alkoxy, N(R⁹)₂, NR⁹C(O)R⁹, C(O)R⁹, oxetane and THF;
R⁷ is H or C₁-C₃ alkyl optionally substituted with one or more halogen;
R⁸ is H or C₁-C₃ alkyl; and
each R⁹ is independently H or C₁-C₃ alkyl, halogen or haloalkyl.

8. The method of any of claims 1 to 7, wherein the PRMT5 inhibitor is compound of Formula IIIA: 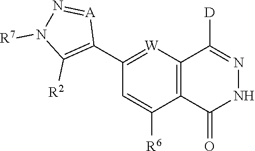
or a pharmaceutically acceptable salt thereof, wherein
A is CR⁹ or N;
D is —CH₂—NH₂, 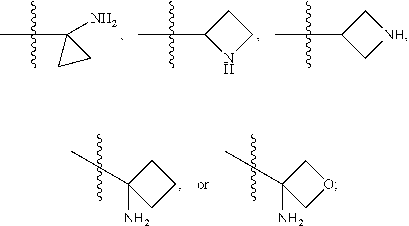 [SMILES: *C1(N)CC1 ; *C1CCN1 ; *C1CNC1 ; *C1(N)CCC1 ; *C1(N)COC1]
W is CR⁹ or N, where R⁹ is H or C₁-C₃ alkyl;
R² is 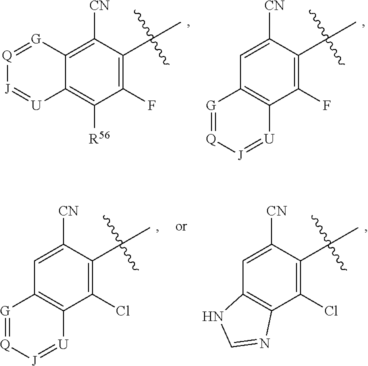
where R⁵⁶ is hydrogen, fluoro, chloro, or methyl,
G, Q, J and U are independently selected from C(H), C(R⁵), and N, provided only one or two of G, Q, J, and U can be N;
each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

9. The method of claim 8, wherein the PRMT5 inhibitor is:

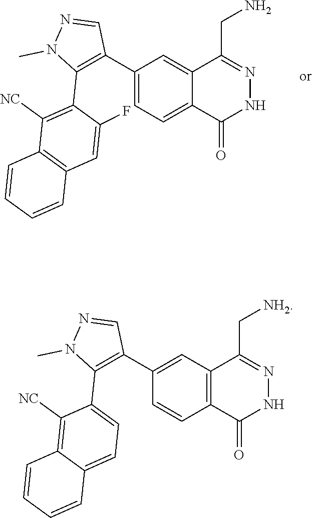 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N]

10. The method of any of claims 1 to 9, wherein the PRMT5 inhibitor is compound of Formula IIIB: 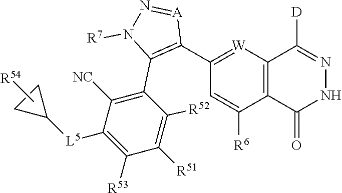
or a pharmaceutically acceptable salt thereof, wherein
A is CR⁹ or N;
D is —CH₂—NH₂, 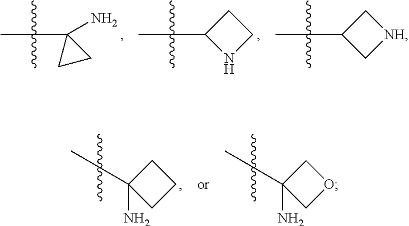 [SMILES: *C1(N)CC1 ; *C1CCN1 ; *C1CNC1 ; *C1(N)CCC1 ; *C1(N)COC1]
W is CR⁹ or N, where R⁹ is H or C₁-C₃ alkyl;
R⁵¹ is hydrogen, fluoro, chloro, or methyl, or R⁵¹ and R⁵² together with atoms to which they are attached form a C₄-C₆ heterocycloalkyl (e.g., hydrofuranyl);
R⁵² is fluoro, chloro, or methyl, or R⁵² and R⁵³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy;
L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl.

11. The method of claim 10, wherein:
A is —CH or —CCH₃;
D is —CH₂—NH₂;
W is —CH, —CCH₃, or N;
R⁵¹, R⁵², R⁵³, and R⁵⁴ are each independently selected from hydrogen, fluoro, chloro, or methyl;
L⁵ is —O—;
R⁶ is hydrogen, fluoro, chloro, or methyl; and
R⁷ is C₁-C₂ alkyl or C₁-C₂ haloalkyl.

12. The method of claim 10 or claim 11, wherein:
A and W are —CH;
D is —CH₂—NH₂;
R⁵¹, R⁵², and R⁵³ are each independently selected from hydrogen, fluoro, chloro, and methyl;
R⁵⁴ is hydrogen;
L⁵ is —O—;
R⁶ is hydrogen; and
R⁷ is methyl.

13. The method of any of claims 10-12, wherein:
A and W are —CH;
D is —CH₂—NH₂;
R⁵¹ and R⁵² are each independently selected from fluoro, chloro, and methyl;
R⁵³ and R⁵⁴ are hydrogen;
L⁵ is —O—;
R⁶ is hydrogen; and
R⁷ is methyl.

14. The method of claim 10, wherein the PRMT5 inhibitor is: 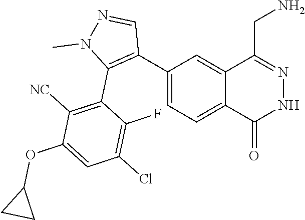 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof.

15. The method of claim 11, wherein the PRMT5 inhibitor is: 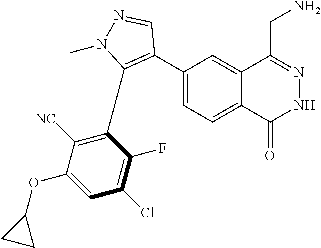
or a pharmaceutically acceptable salt thereof.

16. The method of any of claims 1 to 10, wherein the PRMT5 inhibitor is compound of Formula IIIC: 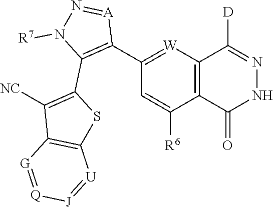
or a pharmaceutically acceptable salt thereof, wherein
A is CR⁹ or N;
D is —CH₂—NH₂, 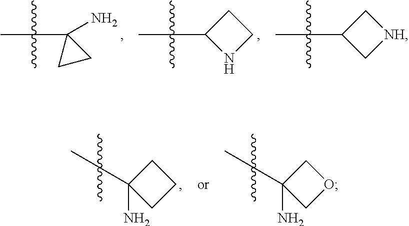 [SMILES: *C1(N)CC1 ; *C1CCN1 ; *C1CNC1 ; *C1(N)CCC1 ; *C1(N)COC1]
W is CR⁹ or N, where R⁹ is H or C₁-C₃ alkyl;
G, Q, J and U are independently selected from C(H), C(R⁵), and N, provided only one or two of G, Q, J, and U can be N;
each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶,
where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl.

17. The method of claim 16, wherein the PRMT5 inhibitor is: 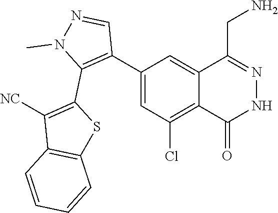 [SMILES: Cn1ncc(-c2cc(Cl)c3c(=O)[nH]nc(CN)c3c2)c1-c1sc2ccccc2c1C#N]
or a pharmaceutically acceptable salt thereof.

18. The method of any one of claims 1 to 17, wherein the therapeutically effective amount of the PRMT5 inhibitor is in the range of about 0.01 to 300 mg/kg per day.

19. The method of any one of claims 1 to 17, wherein the therapeutically effective amount of the PRMT5 inhibitor is in the range of about 0.1 to 100 mg/kg per day.

20. The method of any one of claims 1 to 17, wherein the therapeutically effective amount of the PRMT5 inhibitor is less than 1% of, e.g., less than 10%, or less than 25%, or less than 50% of the clinically-established therapeutic amount.

21. The method of any one of claims 1-20, wherein the BCL-2 family inhibitor is selected from one or more of: ABT-199 (venetoclax), ABT-263 (navitoclax), A-1155463, A-1331852, obatoclax (GX15-070), ABT-737, TW-37, gossypol, (R)-(−)-gossypol, HA14-1, sabutoclax and DT2216).

22. The method of any one of claims 1-21, wherein the BCL-2 family inhibitor comprises ABT-199.

23. The method of claim 22, wherein the BCL-2 family inhibitor is ABT-199.

24. The method of any one of claims 1-21, wherein the BCL-2 family inhibitor comprises ABT-263.

25. The method of claim 24, wherein the BCL-2 family inhibitor is ABT-263.

26. The method of any one of claims 1 to 25, wherein the therapeutically effective amount of the BCL-2 family inhibitor is in the range of about 1 to 500 mg/m² per day.

27. The method of any one of claims 1 to 25, wherein the therapeutically effective amount of the BCL-2 family inhibitor is in the range of about 10 to 300 mg/m² per day.

28. The method of any one of claims 1 to 27, wherein the therapeutically effective amount of the BCL-2 family inhibitor is less than 1% of, e.g., less than 10%, or less than 25%, or less than 50% of the clinically-established therapeutic amount.

29. The method of any of claims 1 to 28, wherein the BCL-2 family inhibitor and the PRMT5 inhibitor are administered sequentially.

30. The method of any of claims 1 to 28, wherein the BCL-2 family inhibitor and the PRMT5 inhibitor are administered simultaneously.

31. The method of any one of claims 1 to 30, wherein the subject previously received or completed a first-line chemotherapy.

32. The method of claim 31, wherein the first-line chemotherapy is gemcitabine, nab-paclitaxel, 5-FU, irinotecan, oxaliplatin, capecitabine, cisplatin, carboplatin, fludarabine, cyclophosphamide, fluoropyrimidine, pemetrexed, doxorubicin, ifosfamide, epirubicin or paclitaxel.

33. A method for treating cancer in a subject, the method comprising administering to the subject:
a therapeutically effective amount of ABT-199 (venetoclax): 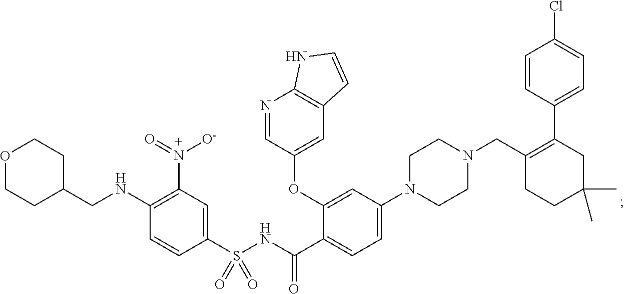
and
a therapeutically effective amount of a PRMT5 inhibitor of formula:

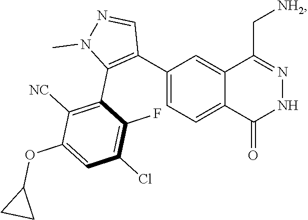

34. A method for treating cancer in a subject, the method comprising administering to the subject:
a therapeutically effective amount of ABT-263 (navitoclax): 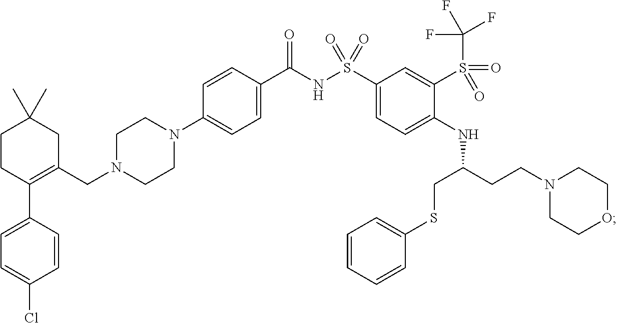 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
and
a therapeutically effective amount of a PRMT5 inhibitor of formula: [chemical structure]
